(19) United States
(12) Patent Application Publication
(10) Pub. No.: US 2024/0079118 A1
(43) Pub. Date: Mar. 7, 2024

(54) eNursing

(71) Applicant: James Kirunda Kakaire, Silver Spring, MD (US)

(72) Inventor: James Kirunda Kakaire, Silver Spring, MD (US)

(21) Appl. No.: 17/803,184

(22) Filed: Dec. 19, 2018

Related U.S. Application Data
(60) Provisional application No. 62/607,513, filed on Dec. 19, 2017.

(51) Int. Cl. G16H 40/20; G06F 21/45; G06Q 10/1053; G06Q 20/32; G16H 10/60

(52) U.S. Cl. CPC G16H 40/20; G06F 21/45; G06Q 10/1053; G06Q 20/3223; G16H 10/60

(57) ABSTRACT

A system for facilitating the process of hiring nurses and other medical workers utilizes a Mobile App and a Website that executes medical job transactions. It processes patient data hence reduces the likelihood of medical errors. The system works in conjunction with a scheduling system to schedule workers. It processes registration of medical workers for short term jobs and registers employers to post jobs. Posted job listings are displayed for workers. Once a worker applies, an alert is sent to the employer that posted the job. The employer views the worker's photo after hiring them and the worker is alerted on their mobile device and email. Employers pay electronically through credit card or bank account and workers are paid electronically. The system which identifies patients by patient identification number, location, sex and age verifies patient data by scanning QR Code or Barcode on patient's arm or bed.

This patent application claims the benefits of provisional application No. 62/607,513, filed Dec. 19 2017 titled eNursing. It is also a continuation in part of patent application No. PCT/US2018/048933 filed on Aug. 30 2018 with confirmation No. 1914.

### FIELD OF ENDEAVOR

This system facilitates the process of hiring nurses and other medical workers without human intervention. It comprises of an authentication module that recognizes the user type and provides only the options accessible by that user. The system provides a user menu for the medical employers to post short term jobs, hire nurses, schedule them and process administrative tasks. It also provides a menu for medical workers that are job seekers to setup a profile, apply for the posted jobs, and manage taxation and electronic payment options. The system processes all necessary human resource without human intervention. The nurses view hours worked and payments in real time; and process patient data from a mobile app resident on mobile devices when hired hence reduce the likelihood of medical errors at work.

### BACKGROUND OF THE INVENTION

This system referred to as eNursing is based on a Mobile App and website that are fully automated to accomplish the tasks involved in the hiring and disbursement of wages. It enables hired medical workers to execute their day to day job functions hence reducing the likelihood of medical errors. The system also works in tandem with the scheduling system to avail worker schedules on an installable mobile app.

The system works in conjunction with the Business, Jobs and Religious Apps Framework (BJRAF) to generate button links dynamically so as to automate processes. The BJRAF provides several functions including user menus, time tracking, invoicing, a payment system, tracking of users on a billing account, employee messaging, a built in HR System, taxation and worker ratings.

### SUMMARY

A system for facilitating the process of hiring nurses and other medical workers electronically utilizes a Mobile App and Website to hire medical workers and facilitating their day to day job activities while maintaining their schedules. All functions are accessible through the Mobile App as one interface and the website is a secondary interface for large screens. Patient data processing by the medical workers via the Mobile App reduces the likelihood of medical errors.

The Mobile App and Website processes registration of nurses and other medical workers for jobs. The registrations are directly accessible to employers that post the short term jobs. The Mobile App and Website provides a means for employers to choose nurses and other medical workers for hire directly based on skills, ratings and location set by the nurse. Taxation, data entry and payments are processed electronically.

The medical workers include but not limited to Registered Nurse (RN), Licensed Practical Nurse (LPN), Certified Nursing Assistant (CNA), Generic Nursing Assistant (GNA), Home Health Aide (HHA), Medical Transcriptionists, Baby Sitter (BS) and Medical Assistants. It helps them accomplish the day to day activities at the jobs and reduces the likelihood of medical errors if their employer participates in the program. The system also works with the Scheduler System to provide a scheduling mechanism for healthcare workers and manage time off requests. It tracks hours worked and process payments in conjunction with the Business Apps Framework system.

The eNurse system which identifies patients by patient identification number, location, sex and age enables healthcare workers to verify patient data by scanning QR Code or Barcode on patient's arm or bed prior to data update or treatment. Patient's records are also pulled manually by touching the buttons corresponding to the patient's ID and service type to provide service and confirm that service has been provided.

### BRIEF DESCRIPTION OF THE DRAWINGS

FIG. 1 Represents the initial User Interface displayed when a Mobile App is launched.

FIG. 2A represents a user interface utilized by nurses and other healthcare workers that have completed account setup and authenticated while FIG. 2B shows a listing a job listing viewed by a medical worker when they hit the job button 5 to apply for a job.

FIG. 3 is a user interface displayed when a healthcare worker presses the Case Load tab on a medium and large screen. It shows the worker's individual case loads.

FIG. 4 is a part of FIG. 3 but displays case details instead of case load. Here a nurse or healthcare worker views details of patients and scheduled patient activities.

FIG. 5 is a continuation of FIGS. 3 and 4 as a tab. It provides a screen for adjusting user settings. However, contents of that tab can be replaced by frequently needed data and have the settings button link somewhere else.

FIG. 6A displays a nurse's case load while FIG. 6B shows case details on small screens for a medical worker providing service such as a nurse.

FIG. 6C displays case load for a prescribing medical worker such as a doctor. Upon opening a button for one patient, contents of FIG. 6D are displayed.

FIG. 7 is the user interface utilized by healthcare supervisors to manager nurses and other activities

FIG. 8 is an algorithm that creates user accounts and authenticates users to process data.

FIG. 9 is a continuation of the algorithm in FIG. 8. It authenticates and enables users to processing workers and patients.

FIG. 10 represents an algorithm that adds patients from the receiving server to the production server in the system.

FIG. 11 is an algorithm that adds patients from the Data Source to the system when local and cloud servers are utilized.

FIG. 12 is an algorithm that authenticates medical users for patient processing.

FIG. 13 is a continuation of the patient processing algorithm in FIG. 12.

FIG. 14 represents the Entity Relationship Model (ER-Model) for a relational database utilized in this system to hire and process medical workers as well as patients. However, a different type of database or file system may be utilized instead of this relational model.

FIG. 15 is a continuation of the ER-Model in FIG. 14. It processes chores, time management and data sharing.

FIG. 16 shows the database schema that results from the ER-Models of FIGS. 14 and 15.

FIG. 17 represents data transmission between user devices and servers in both the local and cloud environments.

### DETAILED EMBODIMENT

This is a system for facilitating the process of hiring nurses and other medical workers electronically. It utilizes a Mobile App and a Website specifically for hiring medical workers and facilitating their day to day job activities and schedules accessible through the Mobile App and website. Patient data processing via the Mobile App and color coded automation reduces the likelihood of medical errors.

The Mobile App and Website processes registration of medical workers for short term jobs. Registrations are directly accessible to employers that post the jobs. The Mobile App and Website provides a means for employers to choose medical workers to directly hire for the short term jobs.

The system which identifies patients by patient identification number, location, sex and age enables healthcare workers to verify patient data by scanning QR Code or Barcode on patient's arm or bed prior to data update or treatment. Patient's records are also pulled by touching the buttons corresponding to the patient's ID and service type to provide service and confirm service delivery.

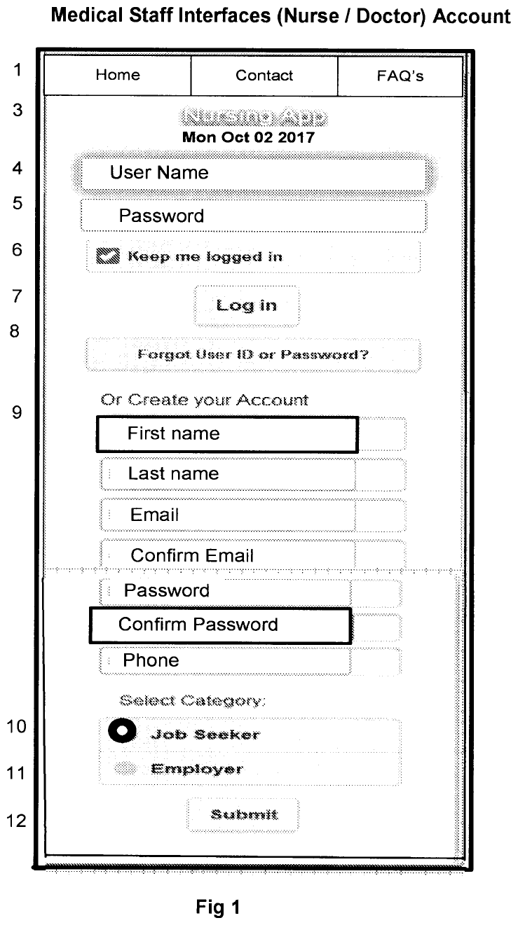

FIG. 1 Represents the initial User Interface displayed when a Mobile App is launched. The top menu 1, comprises of the home page of the app, the contact page and the frequently asked questions page (FAQ's). The Mobile App title 2, is followed by the date 3, user name field 4 and password field 5. To prevent frequent login, the user checks the Keep me logged in box 6. A submit button 7 is labeled Login. To reset a password, the user touches Forgot User ID or Password 8. The bottom part 9 allows a new user to create an account entering their first name, last name, email, password and phone number. They select a category of job seeker 10 or employer 11. A submit button 12 is provided to register the user.

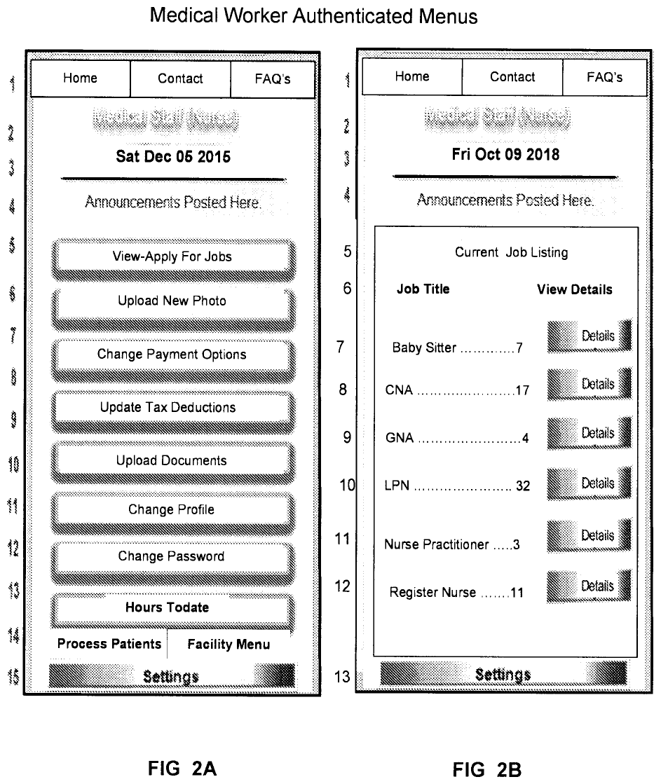

FIG. 2A represents a user interface utilized by nurses and other healthcare workers that have completed account setup and are authenticated. FIG. 2A displays title of the app 2, date 3, announcements 4, a button link for viewing job listings 5, a button link for uploading pictures 6, a button link for changing payment options 8, a button link for updating tax deduction info 9, a button link for uploading other documents such as driver license, social security card copy, resume, paystubs etc 10, a change profile button link 11, change password 12, view hours to date 13, process patients from employer facility 14 and settings 15. A worker can be employed by three facilities at the same time. In such a case, three button links each to one facility data are displayed on the mobile app. However, each button link is activated only at the time when the worker is on duty for that facility.

FIG. 2B shows a job listing as viewed by a medical worker when they hit the job button 5 to apply for a job. A job title is displayed followed by the number of jobs under that title and a button that provides details about the listed job category. Categories listed here include Baby Sitter 7, CNA 8, GNA 9, LPN 10, Nurse Practitioner 11 and Registered Nurse 12. A settings button is provided to configure the app to the likings of the user.

Not showed are buttons for viewing individual schedule, group schedule, requesting time off, medical supplies inventory including drugs and accessories. They also get access to the calendar from the Job and Religious App Framework.

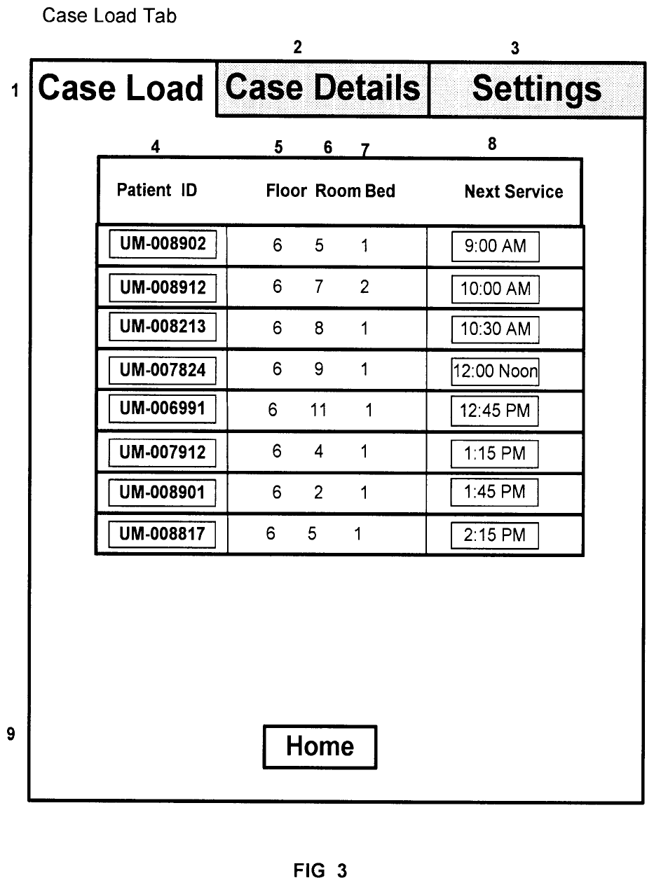

FIG. 3 is a user interface displayed when a healthcare worker presses the Case Load tab on a medium and large screen. It shows the worker's individual case loads 4. It shows the floor on which the patient is located 5, room number 6 and bed number 7 assuming address and building number are displayed. It also shows the time when the patient is supposed to get service 8. The home button 9 returns to the beginning of the application.

Patients' records are identified by record ID 4 to prevent unauthorized personnel from identifying the patients. The medical worker compares the number referred to as patient record ID to the one on the bed or patient arm to identify the patient.

If the worker's device is equipped with a barcode or QR Code scanner, the worker's device scans the code on the patient's arm or bed to assist with identification. When a code is scanned, the patient's record pops up on the worker's device. The scanning feature also prevents misidentification of a patient if the medical worker happens to be tired or absent minded and they can't read the tag on the arm or miss the right button in the application. The medical worker also utilizes the scanner to do patient data entry. Actual times of service and accurate data entry help doctors in analyzing trends related to time, medications and other activities such as blood pressure, body temperature and abnormalities that comes as reactions.

The App further identifies the record by a floor 5, room number 6 and bed number 7. Next service time 8 is also indicated. The Home button link 9 takes the user back to the main menu.

This HIPPA compliant system works on all devices with some kind of operating system. Personally identifying patient information is not sent over mobile devices.

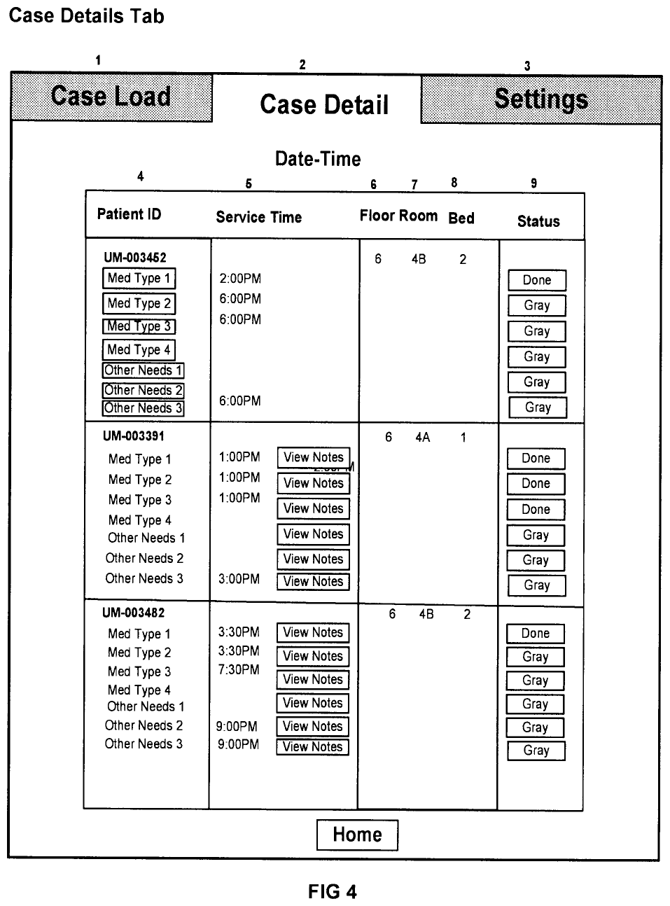

FIG. 4 is a part of FIG. 3 but displays case details instead of case loads. Here a nurse or healthcare worker views details of patients and scheduled patient activities. On a large screen such as that of a tablet, the display shows a patient ID 4, Service Time 5, Floor 6, Room Number 7, Bed 8 and Status 9 in a horizontal layout. On a small screen such as that of a phone, the layout is vertical in 2 to 3 columns.

It is under the Case Details tab that data entry takes place. When a healthcare worker is finished with an item of service, they press the button next to that service and the button toggles to indicate a status of done with a bright color such as light green. No typing is necessary. This prevents data entry errors.

A button is provided alongside each required service to view details of the service in case the medical worker is not familiar with the patient. However, this button is alternatively placed at the button of each patient's record to show all that need to be seen from one button. This conserves space on the device screen. When the worker presses a button to indicate that the assignment is completed (service provided), the button changes color from grey to a bright color such as green.

In another implementation, each service need is a button itself. This is exemplified under patient ID UM-003452 where med Type1, med Type2, medType3 and med Type4 and other Needs are presented as buttons. To view details of a needed service, the medical worker only presses the button for the needed service and views the details in a new pop up window which closes to return to where the worker was. This implementation eliminates the View Notes buttons seen under the other patients. It frees up a column space to make it more mobile friendly.

If a prescription needed at 2:00 PM is named Med Type 1, that name will be a button reading Med Type 1. The worker just presses that button to view details of the drug and frequency. The workers are reminded by alerts that they set on their devices. These alerts can be sound, vibrate or both.

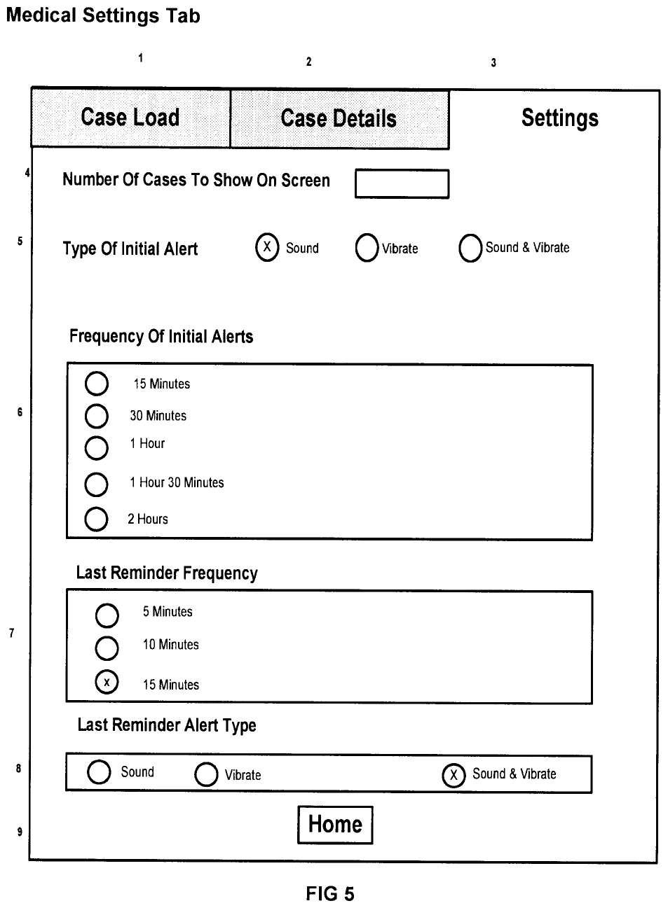

FIG. 5 is a continuation of FIGS. 3 and 4 as a tab. It provides a screen for adjusting user settings 3. However, contents of that tab can be replaced by frequently needed data and have the settings button link somewhere else. The user selects the number of patients to view on the screen 4. They set the type of alert for their mobile app 5 and 8. These include Sound, device vibration or a combination of both sound and vibration. The user also sets frequency of the alerts 6 which could be fifteen minutes, thirty minutes, one hour, one hour and thirty minutes or two hours and last reminder frequency 7. The algorithms executed through the user interfaces, reads registered patients to ensure every patient is assigned a healthcare worker and a treatment plan. Settings reminders triggers alerts so as to provide medications on time. Workers are also alerted when a patient has no assigned medical workers. The actual time data is recorded is considered the service time.

The last reminder gives them the minimum time they can get to the patient and attend to them in a timely manner. The Home button link takes them back to the main menu.

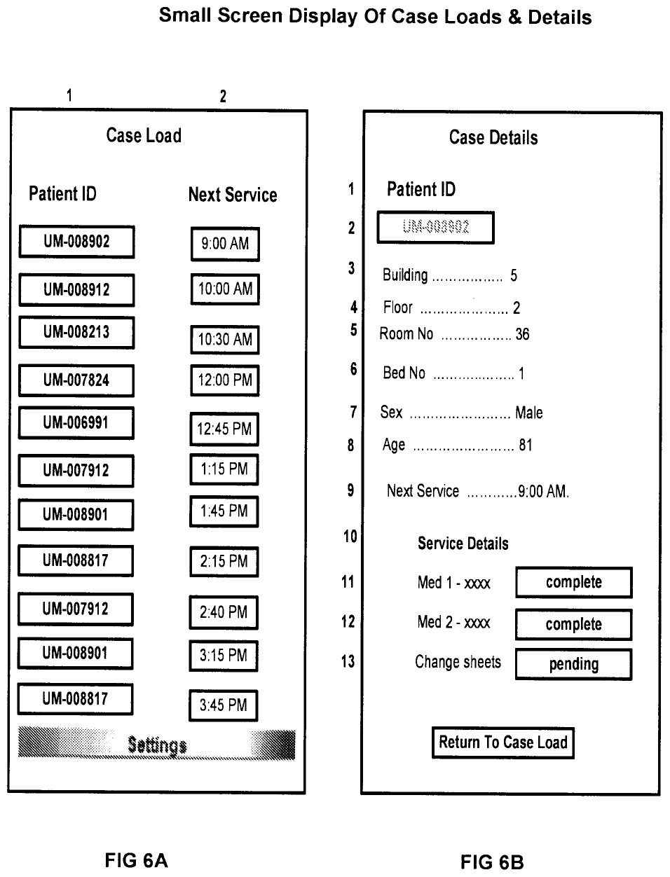

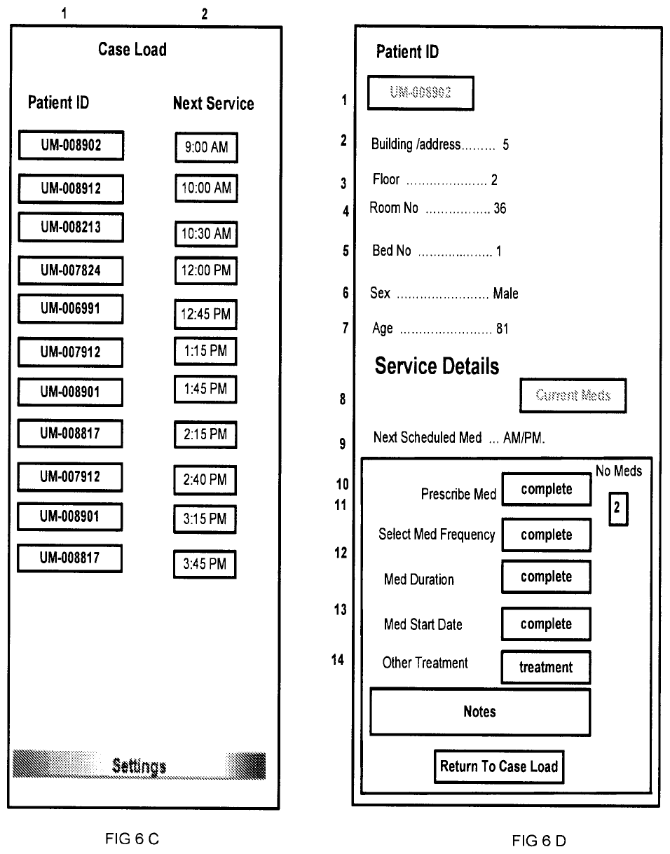

FIG. 6C displays case load for a prescribing medical worker such as a doctor. Upon opening a button for one patient, contents of FIG. 6D are displayed.

FIG. 6A displays a nurse's case load while FIG. 6B shows case details on small screens for medical a worker providing service such as a nurse. It shows a nurse's case load in two columns on a small screen such as that of a mobile phone. Column 1 displays patient ID and column 2 displays the time a patient is scheduled to get the next service. When a user (medical worker) presses the patient ID button 1, it provides details of that particular case as seen in FIG. 6b. The patient ID 1 grays out when details are showed. To prevent misidentification of a patient especially when a medical worker is tired, the system shows the building where the patient is located 3, the floor number and wing 4, Room number 5, Bed number 6, Sex of the patient 7, Age of the patient 8 and the time of next service 9. This is followed by a list of services scheduled for that patient 10. Whenever the medical worker or nurse completes a task 11 and 12, she presses the pending button link and it turns color to show that the task is complete. The word complete or done is also displayed in the button link and data is saved to the server. The unfinished task 13 shows up in a different color such as red or yellow. It is visible to anyone that pulls the record at that time. The button labeled Return To Case Load takes the user back to FIG. 6A.

FIG. 6C is similar to FIG. 6A except that it is meant for a medical user that prescribes medications such as a doctor. FIG. 6D provides step 9 that displays next scheduled med time AM/PM. The medical worker prescribes medications 10, selects medical frequency 11, medical duration 12, med start time 13 and any other treatment 14. A dropdown expands for the medical worker to enter data depending on patient needs. A text area is provided for notes regarding the patient.

This system facilities the process of managing patient records while limiting medical errors that includes but not limited to treating unintended patients, providing incorrect medications, incorrect administration of medications, untimely treatment of inpatients, miscommunication and under communication of providers. It utilizes a mobile app and terminals that employs color coding to distinguish between pending and completed tasks or activities during data entry and retrieval. Patients are identified by their patient identification number, location which includes address, building number, floor-wing, room number, bed number, sex and age. Data processing which defines patients by patient id, address, building number, floor, room number, patient sex, age and service time reduces the likelihood of medical errors.

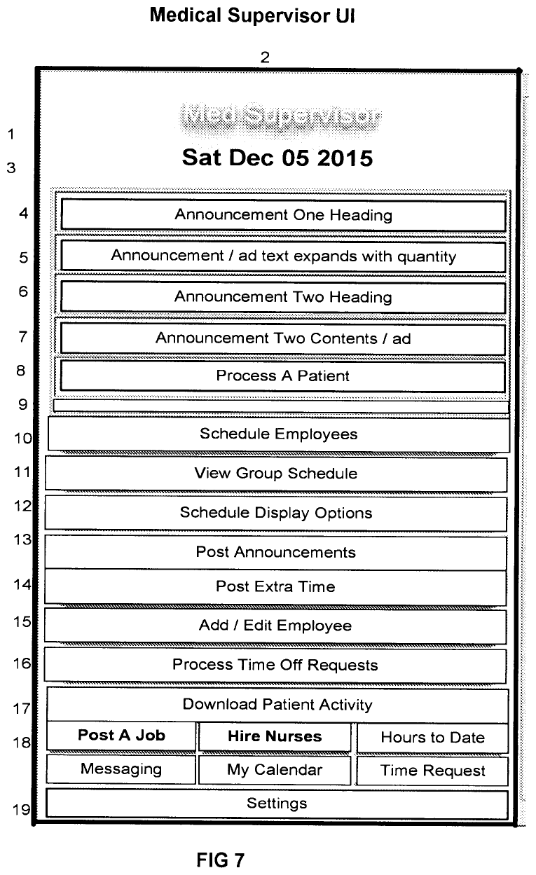

FIG. 7 is the user interface utilized by healthcare supervisors to manager nurses and other activities. A logo not showed is displayed at the top left corner followed by a title 2 which could be company name or department name with a 3D look and fill. Below this is the date of the day 3. Sections 4, 6 and 8 represent announcement headings while 5, 7 and 9 stands for announcement content. The announcements are ads in the form of text, audio and videos obtained from the jobs framework system and the scheduler system. The Schedule employee's button link 10 allows the supervisor to create a new schedule each time. View Group Schedule 11 and allows them to view all medical workers in their groups.

Schedule Display Options 12 shows different options that the supervisor can configure when displaying the schedule.

Post Announcements 13 enables the supervisor to post announcements in the slots indicated by cells 5, 7 and 9. However, the app posts its own ads as announcements at any time. Post Extra Time Available 14 opens up a user interface from where the supervisor posts available overtime.

Add—Edit Employee 15 enables the supervisor to add a medical worker to a facility specific user menu that the medical worker can access to enter data and read about patients. They can also delete workers and edit other worker information. Process time off Request 16 provides the supervisor with ability to retrieve time off requests from workers and grant or deny. This is a feature from the job framework system and scheduler system.

The Download Patient Activity button 17, is pressed to manually download data from the servers that run this application to servers that serves a hospital or other medical establishment. When the button is pressed, the application extracts data from the application's database into comma delimited files or .csv files or data structures such as arrays or array lists. Data is then read from these and uploaded to database tables from where it can be analyzed. However, the process of data transfer is automated so this button link may rarely be used.

The button Post A Job 18 displays a user interface from where the administrator/employer posts jobs. Hires Nurses, Checks Accumulated time or hours to date, use messaging system, utilize the calendar or request for time off. The settings button 19 provides configurations for the app.

This application was designed with HIPPA laws under consideration so no patient identifying information is passed through public networks since personal mobile devices are involved. Patients are identified by their patient ID number.

Our preference is not to identify anyone by name. Initials may be used to represent medical workers in addition to their user id and passwords. Medical workers are identified by user names and emails or actual names depending on facility policy.

When equipped with a barcode or QR Code scanner, a medical worker may get to the patient, open the application and scan the band on the patient or patient's bed. This displays the patient information on the mobile device screen. That prevents a medical worker from matching a patient to a wrong id number.

If a client wants to run statistical analysis on the data with real patient names, they utilize the Download Activity button to get data to a server that can access a database with patient names.

Alternatively, the software is automatically configured to download that data without human intervention. It depends on client's interest. We customize.

Identification of devices that access the application and databases are recorded for security purposes. Identifications may include IP address of the mobile devices or MAC address and are utilized as part of the authentication process.

In summary, data is either posted to the client's server and uploaded by triggers to the receiving server or data is posted to the receiving server and downloaded to the client's accessible machine. This guarantees data availability regardless of network connectivity.

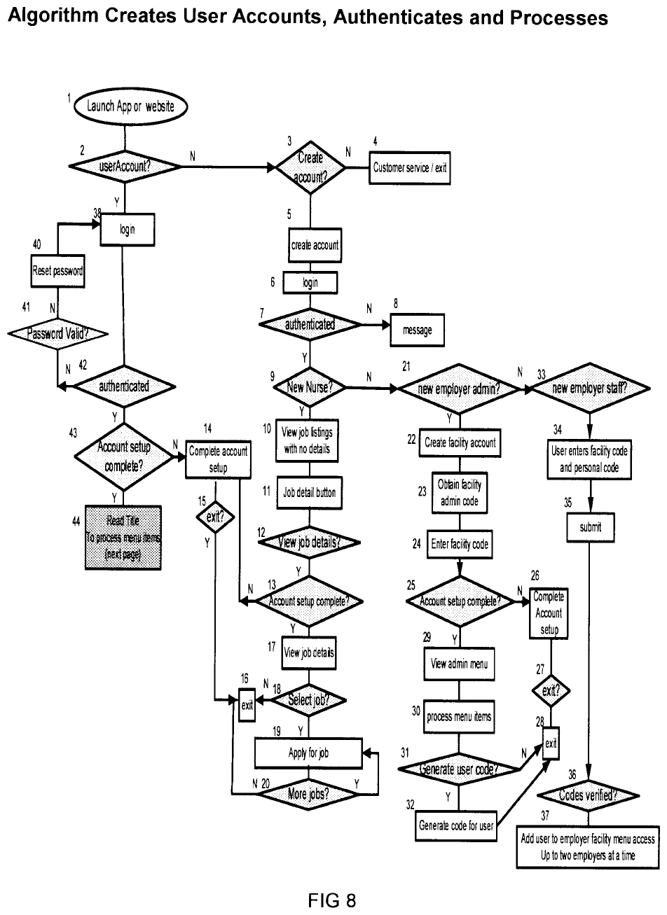

FIG. 8: The algorithms and databases that implements the mobile app in the system of FIG. 8, utilizes a Website for posting medical jobs. It processes registration of nurses and other medical workers for short term jobs. Registrations provide means for employers to choose nurses and other medical workers to hire for the short term jobs the employers post. The employers manage worker schedules; hours worked and conduct other administrative tasks from the system. The system provides a listing of available nurses that apply for the posted jobs to get hired and paid electronically.

They execute an app 1 and checks if the user has an account 2. If the user has no account, they are forwarded to a module 3 that finds out the user's intention. If they don't want to create an account, they are referred to customer service or exit 4. If they want to create an account, a module 5 creates the account for them. After creating the account the user can exit or login 6. If they are not authenticated, a message 8 is provided. Else if they are authenticated, the algorithm reads data storage and compares the user type to provide a corresponding user menu. If the user happens to be a nurse 9, they get a nurse user interface from where they view job listings with no details 10. Buttons 11 are provided to give job details. If the user opens the job detail button 12 to view all the information about a specific job as seen in FIG. 2B, the system checks to ensure the user completed account setup 13 which includes setting up payment option information, tax deductions, identifications documents, job history if any, and a picture that identifies them at a job location when they are hired.

The job applicants sets their wage payment options and tax deductions from the Mobile App when on a mobile device and from a Website when on any device. After completing account requirements, the medical workers also set alerts under their settings options so that they receive alerts when a job is posted or when they are hired by an employer. Both alert options are on by default.

If the nurse or other medical user has not completed account setup, the algorithm sends them to complete the setup 14 or exit 15 and 16. If account setup is complete, opening a job detail button displays job details 17. If the user doesn't select a job 18, they exit else they select a job 19 and apply. They can apply for more jobs 20-19 or exit 16.

If the user happens to be an employer or a facility administrator 21, they create a facility account 22 and obtain a facility administrator code 23 which allows them to manage other users. They enter the facility code 24. If the account setup which includes registering the employer identification number and setting payment options is not completed 25, the employer is referred to step 26 from where they complete their company account setup or exit 27-28.

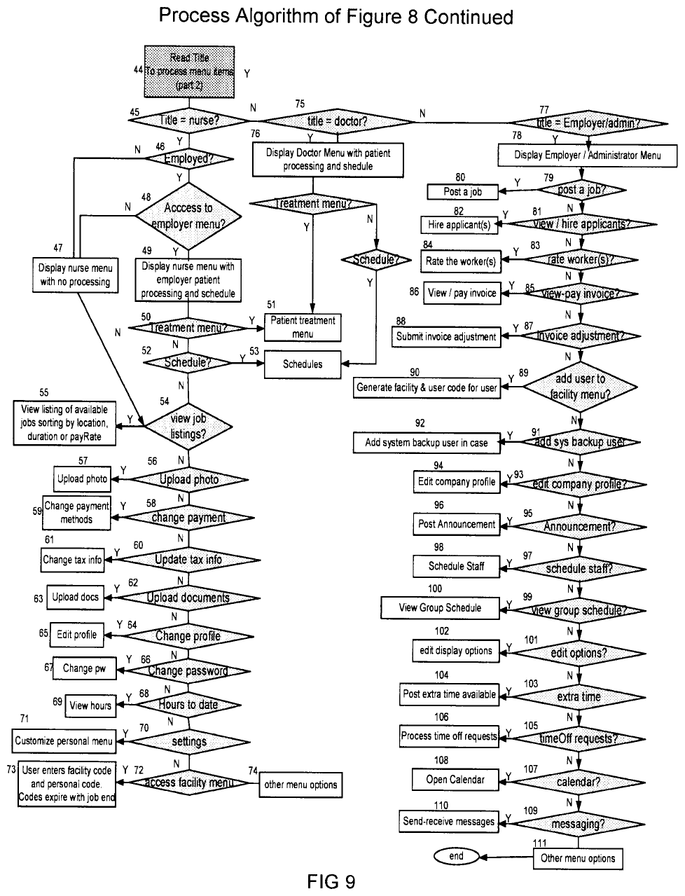

If the employer/admin completed the account setup, they view the admin menu 29 and process the menu options as seen in FIG. 9. If they wish to generate a code for a medical worker 31, they press a button 32 and a code is randomly generated and associated with the worker whose id is entered.

Each facility is identified by a facility code which is appended to the user code to authenticate the user for a specific facility. Upon logging on, the worker gets access to that facility. A medical worker can work for at most three facilities such as hospitals owned by different companies.

An authenticated user gets access to a facility menu only when they are scheduled to work that day and time. The facility menu button for the facility is automatically activated based on worker's schedule. The user can not have access to patient data in any other facilities where they are not scheduled to work at the login time. At their scheduled time, the button link for the facility where they are scheduled is automatically activated.

If the user is not an employer or facility administrator but other employer staff 33, their id is associated with the facility code and their personal code which allows them to access whatever data they are given access to 34-37.

If at the initial logon 2, the user had an account 38, they log onto the system. If the logon fails, they have the option to reset the password 41-40 and logon again 38. If authenticated 42, the system checks to ensure account setup is complete 14 or exit 15-16. If account setup is complete 43, the user is referred to module 44 to process menu items as seen in FIG. 9.

FIG. 9: Under the algorithm of FIG. 9, an authenticated nurse 45 that is not employed by any company 46-47 displays only the nurse menu with no employer facility menu button links. The system provides a user interface for nurses to view job listings and apply for the listed jobs by the various nursing titles including Baby Sitter, CNA, GNA, LPN, RN and NP (nurse practitioner).

If they want to view job listings 54, they view the jobs 55. If the authenticated nurse is however employed, 48, the algorithm displays a nurse menu plus any facility menus where the nurse is employed and scheduled at that time 49. If they want access to the treatment menu 50, they can process employer patients as needed 51. If what they want is to access the schedule 52, they get the schedules for that facility 53. If they choose to upload a photo that identifies them at a job location 56, they accomplish that 57. Both the employed and unemployed nurses get access to uploading photos 56-57, changing payment options 58-59, update tax info 60-61, upload documents 62-63, change profile 64-65, change password 66-67, check accumulated hours to date 68-69 and customize personal menu under settings from where they also set alerts 70-71. Only employed nurses have access to menus of the facilities where they are employed 72-73. Other menu options are provided 74. The system provides a user interface with a button link for nurses to process patients and another button link for accessing the employer's facility menu. They also set reminder alerts for next service as scheduled. An administrator also has ability to set service alerts for all nurses centrally so that each patient is attended to based on the centralized alerts that are individualized for each patient.

The algorithms that execute the Website and Mobile App in this system processes registration of nurses and other medical workers as job applicants for short term jobs. The job applicants sets their payment options and tax deductions from the Mobile App when on a mobile device and Website from any device.

The Mobile App and Website provides means for nurses to change their profile, upload photos that are utilized for identification at a jobsite. These become visible to the employer after hiring the nurse. The nurse or medical worker further upload documents required by employers for identification and work status. They also view accumulated hours to date via a button link.

An authenticated doctor 75 displays a physician menu 76 from which they execute the patient processing menu availed to them with scheduling 76-51 or 76-52.

If the authenticated user is an employer/administrator 77, they display the employer menu 78 from which they post jobs 79-80, view-hire applicants 81-82, rate workers 83-84, view-pay invoice 85-86, submit invoice adjustment 87-88, and add user to facility menu 89-90. This option provides a facility code to the hired user to enable them to access a user interface of that facility. The admin also adds system backup user 91-92 that acts in their capacity, edit company profile 93-94, post announcements 95-96, schedule staff 97-98, view group schedule 99-100, edit display options 101-102 and edit extra time available 103-104. Here user posts over time and makes changes to it. It shows in the nurses mobile app from where they can apply for the over time. Processes time off requests 105-106. Edit-view calendar 107-108, messaging 109-110. Other menus are added 111.

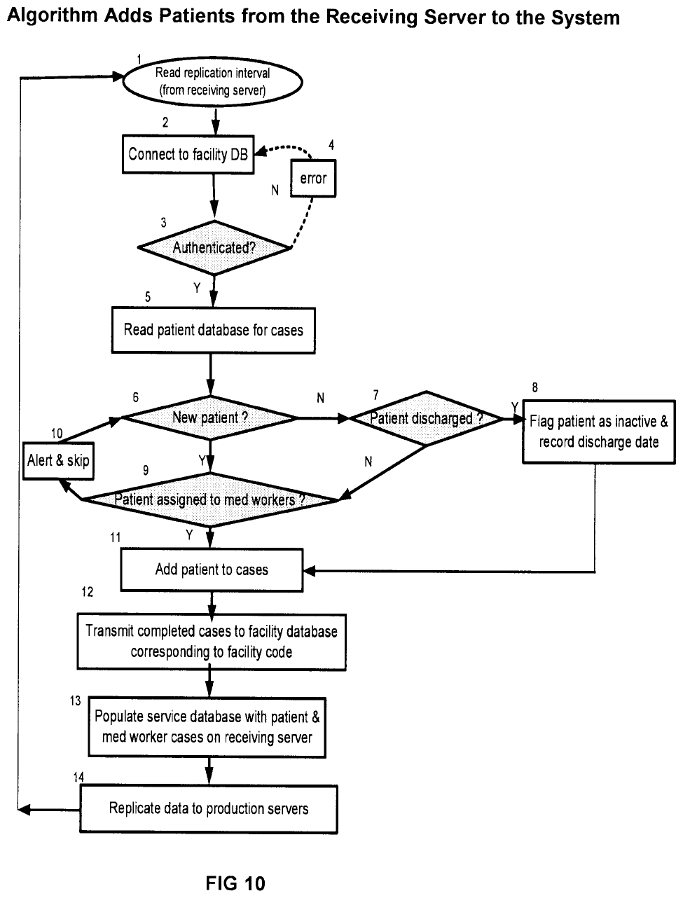

FIG. 10 is the algorithm that adds patients to the software system from the receiving servers. In one implementation, every data item received is replicated immediately. In another implementation where dedicated servers are utilized, data replication interval is set to time t where t equals 15 minutes, 30 minutes, an hour or some other value. The algorithm reads the set interval 1, and connects to the database server when the interval time matches 2. If not authenticated at 3, it keeps trying for a number of pre-set times and finally gives an error message 4.

If authenticated at 3, the algorithm reads the patient database at the origin server traversing through all records 5 looking for new additions 6 and existing patients 7. If existing patients are discharged 8, the patient is flagged as discharged and the discharge date is noted prior to transmission 8. The patient is now added to the case load to be transmitted to the receiving server 11.

If the patient at stage 6 is a new addition, the algorithms executed through the user interfaces in the system reads registered patients to ensure every patient is assigned a healthcare worker and a treatment plan. The workers include doctors and nurses as needed. 9. If no medical workers assigned, the application software alerts the medical stuff by placing a visible icon with a yellow or red color that may even flash for attention 10. The patient that is not assigned medical workers is skipped until attended to. After all assigned patients are added 12, data transmission takes place to a database identified by the facility code involved. The alerts set under 10, execute reminder code that triggers for providing medications on time and alert when a patient has no assigned medical workers. When a medical service is recorded, actual time at which the service is provided is reflected.

The transmitted data populates the database on the receiving server with cases where patients are assigned to medical workers and their respective details 13. Data is then replicated to the production server 14. If however, one server is utilized as the receiving and production server, replication is not necessary except for backup.

In another implementation, a trigger is set instead of a set interval. The trigger listens to changes in the data entry and replicates immediately. It replicates additions and deletions or discharges.

The machine placed at the client's site may be populated with details such as patient's names and medical worker names but incoming data populates the remaining fields. The application auto synchronizes the data. This is a security feature to prevent passing unencrypted identifying information over public networks. Diagnosis, medications and patients are linked by patient ID number not name.

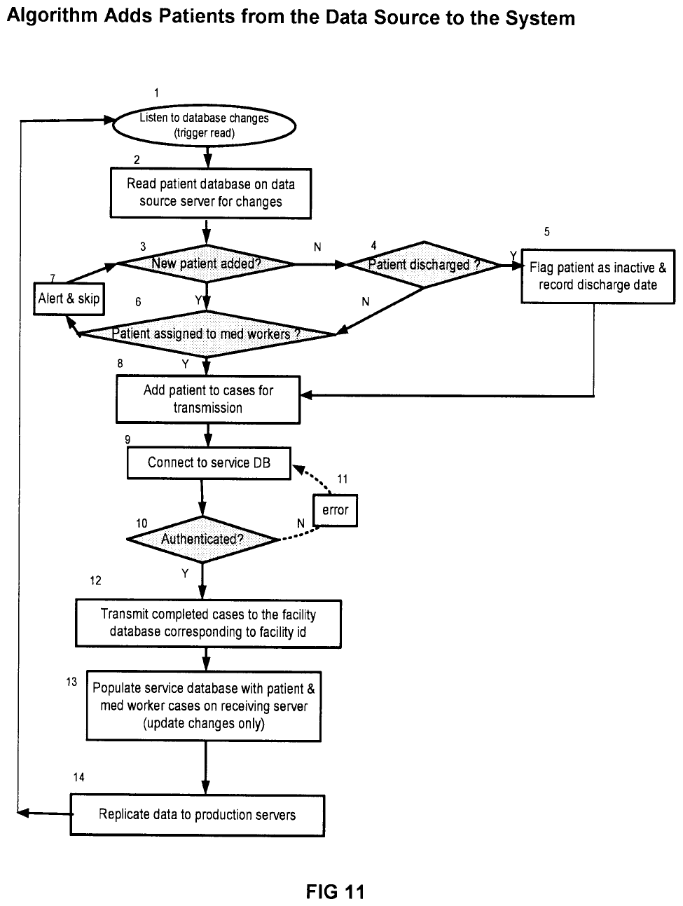

FIG. 11 is the algorithm that adds patient data to the software system from the data source servers that nurse don't connected to via the public internet. The difference between the algorithm of FIG. 10 and that of FIG. 11 is that the later resides on the facility server from where data originates. It is setup with listeners that trigger reading action when a change is detected. The change is replicated to the receiving server in real time without waiting for time intervals to upload data from source to destination (receiving server). The receiving server then provides data to nurses for use in the field.

The application software listens to changes in the database 1. When a new patient is added or an existing patient is discontinued, a trigger invokes the application to read the database on the data source server 2. If the change at step 3 is not a new patient addition and it is a discharge of an existing patient 4, the application software loads the data record and flags the patient as discharged 5. It carries with it the discharge date for reference.

If at step 3, the change is a new patient addition, the application checks to see if the patient is assigned medical workers such as doctors and nurses or drug givers 6. If the patient is not assigned a medical worker, the application software alerts the application users by displaying a visible icon that is brightly colored and in some instances flashing for attention 7 with a record Id informing what record needs attention. Opening the icon gives more details. That record is skipped until it is attended to. That is when the alert is removed.

If the patient at stage 6 is assigned to med workers, they are added to completed cases ready for transmission 8. The application now establishes a connection to the receiving server database to upload the updates 9. If not authenticated at stage 10, the application tries a few preset times and gives an error message to alert that data is not being transmitted 11.

If authenticated at 10, the algorithm transmits the completed cases to the receiving server database corresponding to the facility id 12. The database on the receiving server is populated with current patient and med workers 13. The updated data is then replicated to the production server 14. The process repeats 1-14.

If however, one server is utilized as the receiving and production server, replication is not necessary in that case. Data is always available at the client's local machine for the client to print out schedules in case the network is down. Stored procedures automate data backups.

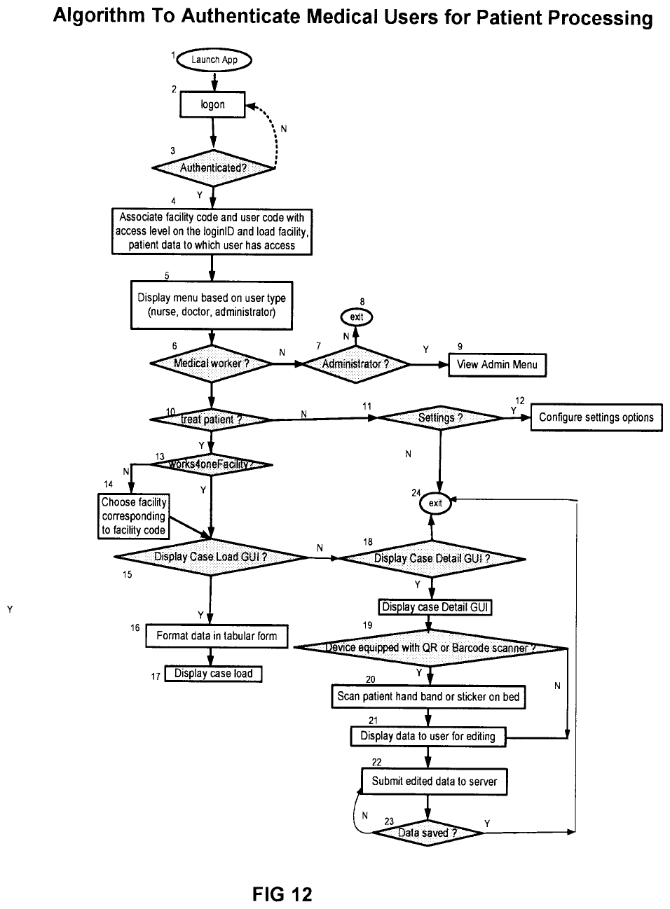

FIG. 12 is an algorithm for authenticating medical workers and processing patients in this system. The system protects patients' records in transit by utilizing record numbers to identify the patients to prevent unauthorized personnel from identifying the patients. Medical workers compare patient numbers to the one on the bed or patient arm to identify the patient. If the medical worker's device is equipped with scanning capabilities, the system identifies a patient's hidden identity by scanning a QR Code, barcode or similar media on the patient's arm or bed to assist with identification and data entry. Scanning the code prevents data entry errors that lead to misidentification of a patient or providing incorrect treatment.

A user launches the application 1 and logs on 2. If authenticated 3, the application reads the database 4 and returns user facility code and type of user and store them in session variables. The algorithm associates access level of the user id to facilities a user has access to and displays the menu corresponding to the user type 5 with the respective facility button links and all the data the user has access to. That is, if the user is a doctor or nurse, a corresponding menu is displayed. If the user is an administrator, administrator menu 9 is displayed.

If the user is not there to process patients 10 or configure settings 11-12, they exit 24. However, if the user is logged on to treat patients or do data entry, the algorithm looks up the facilities to which the user has access 13 and displays their button links in the user interface. The user gets access to the facility data only when they are scheduled to work at that login time. They choose the active facility 14 corresponding to the scheduled facility code.

If the user selects a choice to display case load from the facility menu 15, they get an interface showing formatted patient data 16 in a tabular form 17.

At the facility user interface, if the user decides to display patient details 18, they touch a patient id button and obtain the patient details interface 19 or exit 24.

In order to process one case at a time, the user presses individual patient button links that corresponds to the patient.

If their device is equipped with QR Code or Barcode scanner 19, the worker may scan the patient's wrist band or bed sticker with a code 20 to match the patient code in the application. This code pulls out the corresponding patient record 21 for the worker to edit. It is an added security feature to prevent pressing wrong buttons in case the worker is tired or absent minded. Checking the data prior to treatment or data update also prevents operating on a wrong patient.

Data is now submitted to the server 22. If data is not saved 23, the process repeats until it gives an error message. If data is saved 23, the application exits that particular activity 24.

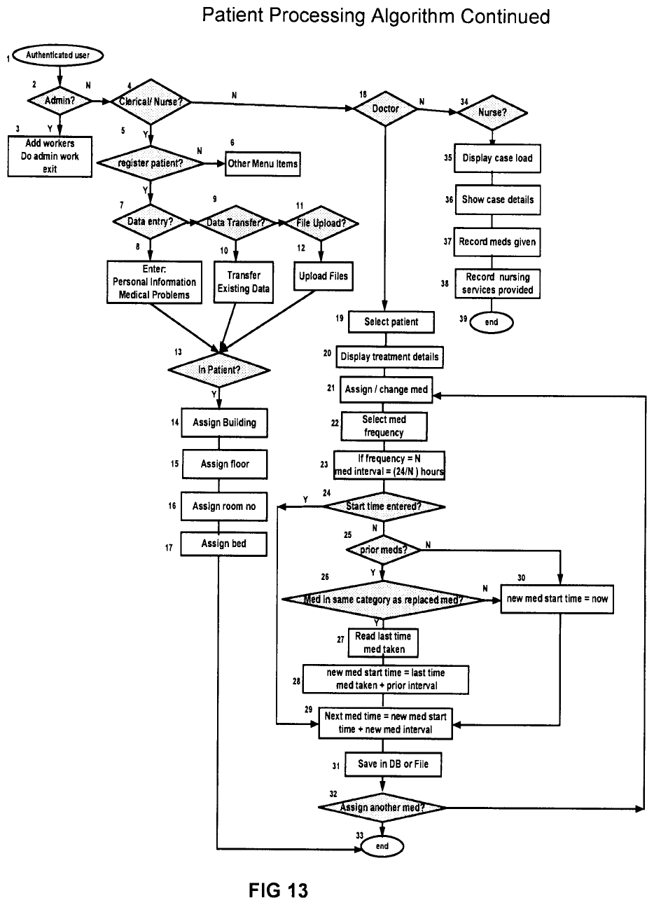

FIG. 13 is a continuation of the algorithm (FIG. 12) that authenticates Nurses and other Med Workers to the system to process patients. The algorithm executes a mobile app in the system and provides a user interface for displaying patient's record by touching button links corresponding to the patient's ID and service type to perform data entry, confirm completion of a treatment or service, or read records. Buttons are color coded and changes color whenever a task is completed.

A nurse or medical worker can be added by their supervisor through an automated process or they can add themselves by filling out a registration form and entering a facility and personal code provided to them by their employer/supervisor. An authenticated user 1 is tested for user type 2. If an administrator, they get the admin menu from which they add workers and perform other admin tasks 3. If the user is of type clerical or nurse 4, they have the option to register patients 5 or perform other tasks on the menu 6. If they are logged in to register patients, they perform data entry 7. Here the system identify patients by patient identification number, address, building number or name, floor, room number, patient sex, and patient age 8. The system provides the service time in a button for easy data entry. Data processing which defines patients by patient id, address, building number, floor, room number, patient sex, age and service time reduces the likelihood of medical errors.

The algorithm provides the option to transfer data 9 utilizing a user interface button 10. It also provides an option for file upload 11 with a command to accomplish that 12. If the person registered is an in patient 13, a building 14, floor 15, room number 16 and bed number 17 are assigned in addition to the address which is auto filled.

The algorithm in the system automates the times of next service by setting medical start time and utilizing medication frequency times in conjunction with start time of the initial dosage and time intervals to compute the next times medication is to be administered. The times are loaded in button links that are displayed for nurse's in a user interface for touch execution. The intervals serve as references to remind nurses when to give meds next.

The system provides button links for selecting med administration frequency which includes Once a day, 2 times a day, 3 times a day, 4 times a day, Weekly, Biweekly, Monthly, Quarterly, semi-annual and annually. A prescribing party opens buttons and selects med times which are accessible to the providing nurse via a graphical user interface with button links.

Furthermore, the algorithm in this system identifies prescription drugs that are about to expire and label them providing a color coded button link for physicians to renew, replace or discontinue the prescription. An alert is automatically sent to attending physicians until they turn it off to indicate that they have worked on it. The button link then changes color and the record reflects a new med.

If the user at login happens to be a doctor 18 or anyone that diagnose and prescribes, they get a special menu from which they select a patient by pressing a button or scanning a QR Code/barcode 19. A menu option displays treatment details 20. The medical worker diagnoses and prescribes, changes or discontinues medications 21. When medication is assigned to a patient, the medical worker selects frequency of medication administration 22. If frequency N is in terms of times in a day 23, the algorithm divides the number of hours in a day (24) by N to obtain the interval or length of times between administrations of the medication. Med frequency can also be once a day, once a week, once a month, quarterly, annually or as desired by the prescribing med worker.

If start time is not entered 24 and there are no prior meds 25, new med start time is assigned to now 30. If start time is not entered 24 and there are prior meds 25, the system checks to see if the medication is in the same category as the prior med 26, reads the last time med was taken 27 and assign the med start time to last time med taken+prior interval 28.

If med start time is entered at step 24 or obtained by calculations through steps 25-28, the next med time is provided by new med start time+new med interval 29. Data is then saved to a database, file or other 31. If the medical worker wants to assign another med 32, they repeat the process 21-32 else they exit 33.

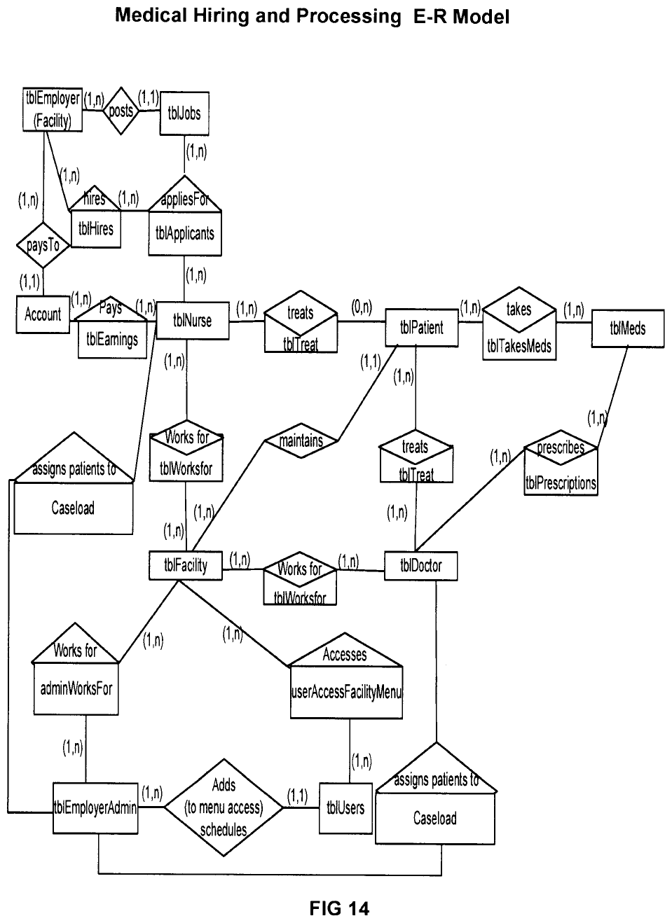

FIG. 14 represents the Entity Relationship Model (ER-Model) for a relational database utilized in this system. However, a different type of database or file system may be utilized.

The E-R Model of FIG. 14 pertains specifically to workers in a clinical setting where they deal with medications and other similar items. The settings include but not limited to hospitals, nursing homes, prisons, shelters and schools.

The entity tblEmployer stores data pertaining to Employers that posts jobs in the entity tblJobs. The nurses in the entity tblNurse apply for the jobs posted in tblJobs. They become job applicants and are stored in the entity tblApplicants as available workers. The entity tblApplicants is a runtime entity or view. Data stored in that entity is only temporary and can be stored in a data structure instead. The entity tblHires store medical workers that are hired for the job after they apply. The employer in the entity tblEmployer pays wages to an account with the entity Account. The account then pays to the worker in the entity tblNurse.

The entity tblMeds is the record of all medications. It has various attributes including category of medication. The entity tblDoctor represents physicians that diagnose and treat patients. When they do, all patient activity record that includes diagnosis, instructions for taking meds (frequency and dosage) extra are stored in the entity tblTreats. On the other hand, prescriptions are stored in the entity tblPrescriptions.

The prescription medicines are classified and placed into categories for treating patients. Each prescription is related to at least one illness. The system automatically detects the medications based on diagnosis entered. The system recommends what drugs to give to the patient based on the drug or medication category and recorded symptoms. Prescription medicines in a category are automatically detected as a replacement of a prior medicine and medications administered to a patient are recorded at actual service delivery time.

Patient med intake records are stored in the entity tblTakesMeds. Data entered by a nurse from a mobile device becomes visible to all providers instantaneously.

The entity tblNurse represents workers that cares for patients and gives them medications and all other needs. These workers could be registered nurses (RN), LPN, HHA, GNA, CNA, Nurse Practitioner or any person entrusted to administer medicines to patients. Similar to the doctors, nurse treatment records are stored in the entity tblTreats.

When developing the database, the entities tblDocter and tblNurse may be combined into one entity tblMedWorkers for faster processing. The medical workers are distinguished by their titles in this entity.

The entity tblPresciptions keep records of medicines that are administered to patients. Every nurse or medical worker is assumed to work for at most three facilities. Work records are stored in the entity tblWorksfor with a composite key. Employer Admin that is assumed to work for a facility assigns nurses or medical workers to facilities. The admin generates nurse code and provides it to a nurse along with a facility code. Medical workers added to facility interface are stored in the entity userAccessFacilityMenu entity which has as some of its attributes userId, facilityId, EmployerAdminId, (one that gave the user access) facility code, user code and notes. The Employer Admin also assigns patients to medical workers such as doctors and nurses and these assignments are stored in the entity Caseload.

The entity tblPatient, represents a sick person that is provided with treatment and given medications. It contains an attribute named status or patientStatus which is defined as active, discharged or deceased. It helps to maintain the number of cases assigned to each health care worker. It is updated when a patient is discharged so that the discharged patient doesn't show up under anyone's cases. Triggers are set on this entity so that each time a new patient is added or an existing patient is discharged, the trigger activates the application to read the database and send updates to the receiving server that feeds the production server. The Mobile App and website interacts with production servers.

The application software authenticates users to enter and retrieve data in the database that is generated from these entities. Alternatively, data is stored in files other than databases.

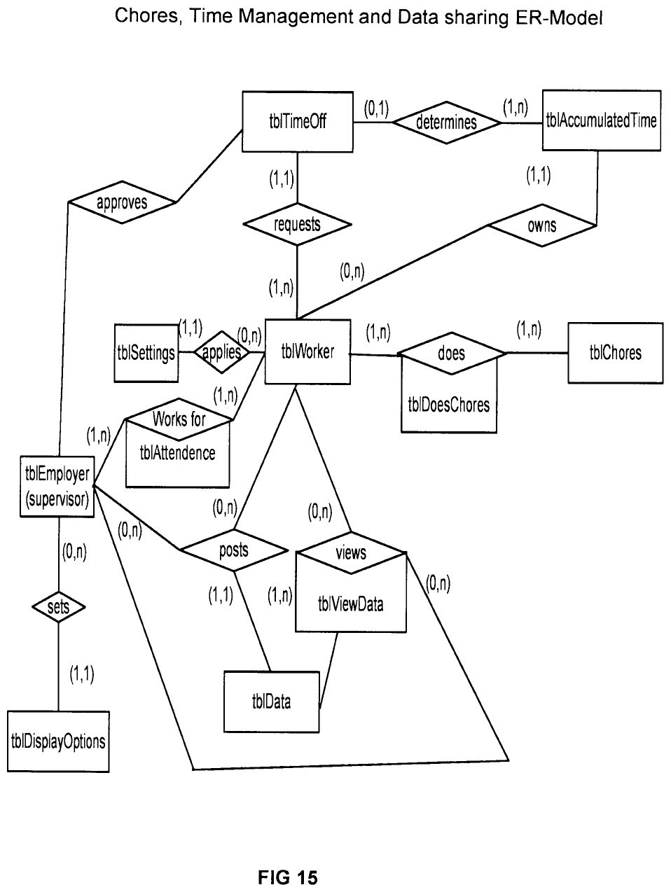

FIG. 15 is a continuation of the ER-Model in FIG. 14. It depicts the other major database entities utilized in the system and how they relate to each other. The entity worker stores the medical worker's profile. The worker works on various assignments. They do chores and the data regarding chores is stored in the entity tblChores. The activity between the chores and worker are recorded in the entity tblDoessChores. Workers accumulate time which becomes their property. The accrued time is stored in the entity tblAccumulatedTime. When the workers request for time off, their electronic request is stored in the entity tblTimeOff. The time they get off is determined by the accrued time in the entity tblAccumulatedTime.

The worker works for employers whose data is stored in the entity tblEmployer. The worker's attendance is stored in the entity tblAttendence which shows the hours worked and all necessary attributes that relate to payments. The tbleAttendance entity is linked to the tblAccount entity not showed. It is the tblAccount entity that stores wage payment data. The employers or supervisors sets display options on the mobile app and website and these are stored in the entity tblDisplayOptons. Medical workers posts data to entities pertaining to patients and are represented by tblData in this diagram. Activity of posting data is stored in the entity tblViewData. Both workers and employers view and use the data collected. Regarding patients, the data is utilized to analyze time related trends including body reactions to treatments and demography of the kind of illness involved. Each patient is recorded with the exact area of residence which includes address and zip code. A count of patients is incremented by one for every treatment provided. The number of people with a given disease in a specific area can help during data analysis to point to cause of the disease.

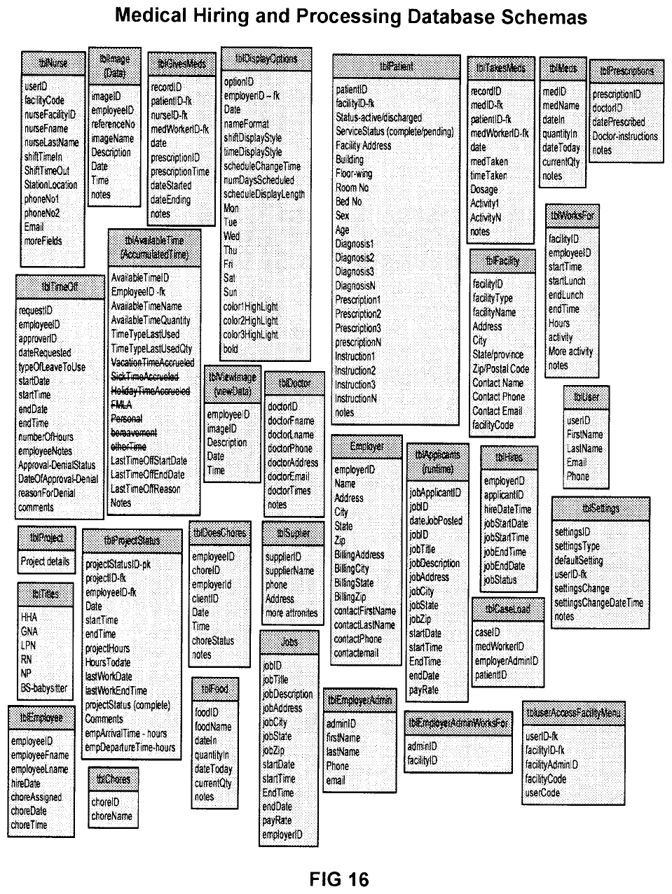

The database schema of FIG. 16 represents the medical E-R Model of FIGS. 14 and 15. The entity tbNurse stores profiles of all medical workers other than doctors. They are distinguished by titles such as RN, LPN, CNA, GNA, Nurse Practitioner, Baby Sitter and so on. The entity tblImage stores data in form of images and their descriptions. The entty tblGivesMeds is represented in the schema as tblTreats. It stores nurse-patient treatment records.

The entity tblDisplayOptions stores application display options set by the employer. The attributes for the entity includes employerID, date option set or changed, name display format, shift display style, time display style, schedule change time, number of days in a schedule, schedule display length, days of the week Monday-Sunday, colors used to highlight and other style formatting.

The entity tblTakesMeds records med intake records. Some of the major attributes are: med ID, patient ID, med worker ID, date, med taken, time of med, dosage, activity 1, activity 2, activity N, notes. As showed with the attributes, the entity tblMeds stores information about meds and tblPrescriptions stores data regarding activity on each of the prescriptions.

The entity tblTimeOff stores information about employee time of requests. The major attributes includes time off requested, employeeID, approvalID/(denialID, date requested, typeOfleaveToUse, start date, start time, end date, end time, number of hours, employee notes, approval denial status, date of approval/denial, reason for denial, comments. This is part of the Jobs, Religious & Political Framework System

The entity tblAvailableTime stores the different times a worker has accumulated in terms of hours. The main attributes are as showed in the table on FIG. 16. The entity tblDoctor stores doctor profile. The main attributes are as seen in the table in FIG. 16.

The entity Employer stores employer profile as seen with the major attributes listed. Similarly, the entity or view tblApplicants stores available workers that have applied or responded to job postings and are waiting to be hired. Once they are hired, they are placed in the entity tblHires with the main attributes listed as jobID, employerID, applicantID, hireDateTime, jobStartDate, jobStartTime, jobEndTime, jobEndDate, jobStatus, notes.

In the entity tblWorksFor (tblEmpCompany), the company id, the facilityID or code allows a user to access data from that facility only. The user type code allows a user to access data pertaining to their patients only or their work section only and when they are scheduled to work. They don't get access to data on their days off.

The facility attribute in the tblPatient entity identifies the facility such as hospital or nursing home where a patient comes from and the particular branch. The facility attribute used together with the employee ID in relation to the tblFacility entity enables the employees such as doctors and nurses to access records of their patients from the tblPatient entity.

That way, employees only access patients they are assigned to. During the implementation phase, all medical workers may be placed in one entity named tblWorkers or tblEmployees and identified by job title.

The entity tblTimeOffRequest is utilized to process and track employee time off. It contains the attributes including request ID, employee ID, date of request, type of leave request, start date of leave, start time, end date, end time and number of hours calculated by the application system. When an employee submits a time off request, it is saved in this entity. It becomes visible to the manager who then approves or denies. The manager has a space for writing a reason for denying the request and this space is represented by the attribute reason for denial.

The rest of the entities store data as showed by the attributes and are utilized based on the relationships as shown by foreign keys after normalization. The database schema is customization to fit licensee needs. They are not written out here to conserve space and limit the size of the patent application.

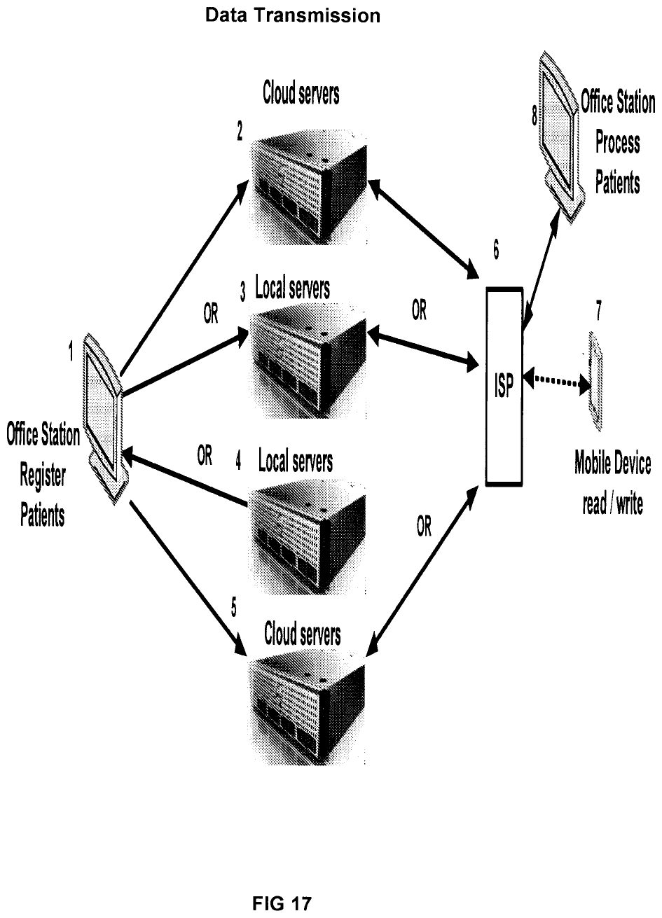

FIG. 17 represents Data Transmission that takes place in the system. The system, accessible over terminals connected to a network wirelessly or by wires, implements a mobile app and touch screen technology is utilized to process patient data transmitted to and from cloud environments. Data is locally modified to limit it to description of patients. Office stations 1 registers data to cloud based servers 2 or local facility servers 3. Data is replicated to production servers 4 and accessed by all terminals. In another implementation, a terminal 1 records data to cloud servers 5. A cloud based production server avails the data for access. Getting through an Internet Service Provider (ISP) 6, mobile devices 7 and remote terminals 8 read and write data by authentication. Data in transmit is altered so that it is not accessible to unauthorized personnel. In one implementation, all data is saved to a local facility server then replicated to cloud based server. In another implementation, data is saved to the cloud then replicated to local servers for storage.

1-20. (canceled)

21. A system for facilitating hiring and electronically paying nurses and other healthcare workers, the system comprising:
at least one server with a processor coupled to a memory, the memory for storing a database system, a file system and instructions executable by the processor to cause the system to perform operations relating to creating a user account for a healthcare job seeker and a user account for a healthcare employer, wherein the healthcare employer utilizes the user account for hiring and paying the healthcare workers, managing time, processing and sharing data;
a computer system configured to execute a website for creating the user accounts utilized for seeking healthcare jobs, for hiring and electronically paying the nurses and other healthcare job seekers or workers and for data processing;
an algorithm for creating user accounts and authenticating the users to process data;
a mobile device with a processor coupled to a memory, the memory configured to store a mobile app, wherein the processor execute instructions for creating the user accounts for seeking healthcare jobs, for hiring and electronically paying the nurses and other healthcare job seekers or workers, and for data processing, wherein the mobile device accesses the website.

22. The system of claim 21, wherein the mobile device is configured to display an interface for creating an account for the healthcare job seeker and for the healthcare employer, wherein the system provides for setting and changing a personal profile, and wherein the personal profile includes a name and at least one of an email and a phone number that is utilized with a password to log into the system.

23. The system of claim 22, wherein the mobile device is configured to register the healthcare workers to apply for jobs, get hired and get paid electronically, wherein the mobile device authenticates the healthcare workers to logon and perform data processing, and wherein the healthcare workers includes at least one of a registered nurse (RN), a licensed practical nurse (LPN), a certified nursing assistant (CNA), a general nursing assistant (GNA), a nurse practitioner, and a baby sitter.

24. The system of claim 21, wherein the computer system is configured to display an interface for creating an account for the healthcare job seeker and for the healthcare employer, wherein the system provides for setting and changing a personal profile, and wherein the personal profile includes a name and at least one of an email and a phone number that is utilized with a password to log into the system.

25. The system of claim 24, wherein the computer system is configured to register the healthcare workers to apply for jobs, get hired and get paid electronically, wherein the computer system authenticates the healthcare workers to logon and perform data processing, and wherein the healthcare workers include at least one of the registered nurse (RN), the licensed practical nurse (LPN), the certified nursing assistant (CNA), the general nursing assistant (GNA), the nurse practitioner, and the baby sitter.

26. The system of claim 21, wherein the mobile device and the computer system are configured to provide for the nurses and the other healthcare workers to upload identifying documents, wherein the identifying documents includes at least one of a driver's license, a copy of a social security card, a resume, a paystub, a work authorization status document, and a photo visible to the employer, wherein the documents are utilized for identification at a jobsite.

27. The system of claim 26, wherein the mobile device and the computer system are configured to provide for the nurses and the other healthcare workers to work at one or more facilities in a time pay period.

28. The system of claim 27, wherein the mobile device and the computer system are configured to process electronic payment options or methods through the website or the mobile app, wherein the electronic payment options or methods, involves paying the nurses and the other healthcare workers by at least one of a direct deposit to a bank account, a pre-paid debit card, an electronic check, and a location pickup.

29. The system of claim 28, wherein the mobile device and the computer system are configured to provide for managing or setting and changing of the payment methods.

30. The system of claim 28, wherein the mobile device and the computer system are configured to provide for the job seekers to manage taxation or change tax deductions through the mobile app or the website.

31. The system of claim 27, wherein the mobile device and the computer system are configured to process human resource (HR) data or paperwork without human intervention, wherein the data is saved to the at least one server.

32. The system of claim 27, wherein the mobile device and the computer system are configured to process the healthcare worker ratings.

33. The system of claim 27, wherein the mobile device and the computer system are configured to provide for hiring of the nurses and the other healthcare workers based on at least one of skills, ratings and location set by the nurses.

34. The system of claim 27, wherein the mobile device and the computer system are configured to provide for the employer or administrator user to add a backup user that acts in capacity of the employer user or administrator on the system to accomplish job tasks of the employer.

35. The system of claim 27, wherein the mobile device and the computer system are configured to provide alerts to the nurses and the other healthcare workers when a healthcare job is posted or when the healthcare workers are hired, wherein the nurses and the other healthcare workers further configures the system in the settings options to set a type of alert including a sound and a vibration.

36. The system of claim 26, wherein the mobile device and the computer system are configured to provide for the healthcare worker to view hours worked to date in real time, wherein the hours worked are viewed from the mobile app or the website.

37. The system of claim 1, wherein the mobile device and the computer system are configured to identify patients by patient identification number, location, sex and age, wherein the data processing defines patients by patient id, address, building number, floor, room number, bed, patient sex, age and service time, and wherein the data processing reduces the likelihood of medical errors.

38. The system of claim 1, wherein the mobile device and the computer system are configured to protect patients' records in transit by utilizing record numbers to identify the patients to prevent unauthorized personnel from identifying the patients, wherein the medical workers compare patient numbers to the numbers on the bed or patient arm to identify the patient.

39. The system of claim 38, wherein the mobile device and the computer system are configured to identify a patient's hidden identity by scanning a QR Code, barcode or similar media on the patient's arm or bed to assist with identification and data entry, wherein scanning the code prevents data entry errors that leads to misidentification of a patient or providing of incorrect treatment.

40. The system of claim 1, wherein the mobile device and the computer system are configured to facilitate the process of managing patient records while limiting medical errors that includes at least one of treating unintended patients, providing incorrect medications, incorrect administration of medications, untimely treatment of inpatients, miscommunication and under communication of providers, wherein the system utilizes color coding to distinguish between pending and completed activities during data entry and retrieval.
